(19) United States
(12) Patent Application Publication
(10) Pub. No.: US 2026/0037855 A1
(43) Pub. Date: Feb. 5, 2026

(54) Training Large Language Model to Analyze Psychological Test Data

(71) Applicant: Equalearning Inc., San Diego, CA (US)

(72) Inventor: Shih-Yuan Wang, San Diego, CA (US)

(21) Appl. No.: 18/789,068

(22) Filed: Jul. 30, 2024

(51) Int. Cl. G06N 20/00; G16H 10/60

(52) U.S. Cl. CPC G06N 20/00; G16H 10/60

(57) ABSTRACT

A method for a web platform to train a large language model platform (LLM) to respond to user inquiries, which includes obtaining a context-data from a data analysis engine, determining a context based on the context-data, selecting a pre-trained context template for the context, determining a system command for the LLM platform, and transmitting the context-data, the pre-trained context template, and the system command to the LLM platform.

### FIELD OF THE INVENTION

The present invention generally relates to computer-assist psychological testing system, and more specifically to an artificial intelligence based system and method for tailoring motivational testing to specific user.

### BACKGROUND OF THE INVENTION

Due to the rapid development of generative artificial intelligence (AI), there are already many applications of large language model (LLM) on the market. These applications mainly focus on collecting and integrating external information. However, in the field of psychology and more specifically in the field of the “motivation science” within the psychology, or personality testing, no one has attempted to use LLMs for analysis.

Many have used generic AI engines, such as ChatGPT, to assist in user testing. However, as it has been reported often ChatGPT hallucinates answers; thus making it not very useful for user testing applications.

The present invention introduces a novel system to use artificial intelligence in the field of psychology and personal testing.

### SUMMARY OF THE INVENTION

The present invention has been made to take advantage of advancement in AI and apply this advancement in the field of psychology or personality testing. The present invention is a cloud-based big data and AI platform capable of analyzing personal psychological test data and integrating data, more specifically the present invention is an AI platform capable of analyzing motivational data. Its product, Motivation Quotient (MQ), helps individuals understand their intrinsic motivations, find suitable learning and career directions, and increase chances of success.

The present invention in one embodiment is a method for an web platform to train a large language model (LLM) platform to respond to an inquiry from a user, the method comprising obtaining a context-data related to user motivation from a data analysis engine, determining a context based on the context-data, selecting a pre-trained context template for the context, determining a system command for the LLMs platform, and transmitting the context-data, the pre-trained context template, and the system command to the LLMs platform.

### BRIEF DESCRIPTION OF THE DRAWINGS

FIG. 1 depicts a LLMs process 100 according to one embodiment of the invention.

FIG. 2 illustrates a MQ analysis process 200.

FIG. 3 illustrates a MQ training process 300.

FIG. 4 is architecture of a system according to one embodiment of the invention.

### DETAILED DESCRIPTION OF THE INVENTION

Many factors had impeded employment of AI in the field of psychological and personal testing. Below are the three most significant factors.
1. The development of LLMs has only recently begun in earnest. For example, ChatGPT 4.0 only started being widely used in 2024. Previous versions, due to limitations in data volume, technology, etc., were not capable of analyzing users' intrinsic motivations.
2. Most personality tests on the market are primarily based on inductive methods. Inductive methods inherently have a limited amount of data and combinations, so LLMs cannot fully leverage its capabilities.
3. The most significant reason is that LLMs itself cannot understand the individual/group psychological conditions of users, and it lacks the systematic knowledge needed to analyze personal psychological conditions.

The third point is often the key to determining whether LLMs can be used to analyze individual or group motivations.

The present invention is a system that takes employs the principle from retrieval augmented generation (RAG) mechanism by dynamically integrating a data from a previously executed user testing during a user inquiry to an AI engine. The system according to the present invention is a cloud-based big data and AI platform capable of analyzing personal psychological test data and integrating data, the data being essentially related to motivational factors for the user. Its product, Motivation Quotient (MQ), helps individuals understand their intrinsic motivations (based on the—Motivation Science), find suitable learning and career directions, and increase chances of success. It promotes self-awareness, enhances learning enthusiasm, and guides individuals to realize their potential through education. On a group level, MQ data enables analysis of the motivational distribution of group members, optimizes task allocation, and improves collaboration efficiency. Knowing what motivates a person helps employees work autonomously, promotes the pursuit of excellence, and improves interpersonal relationships, enhancing team cooperation and management effectiveness.

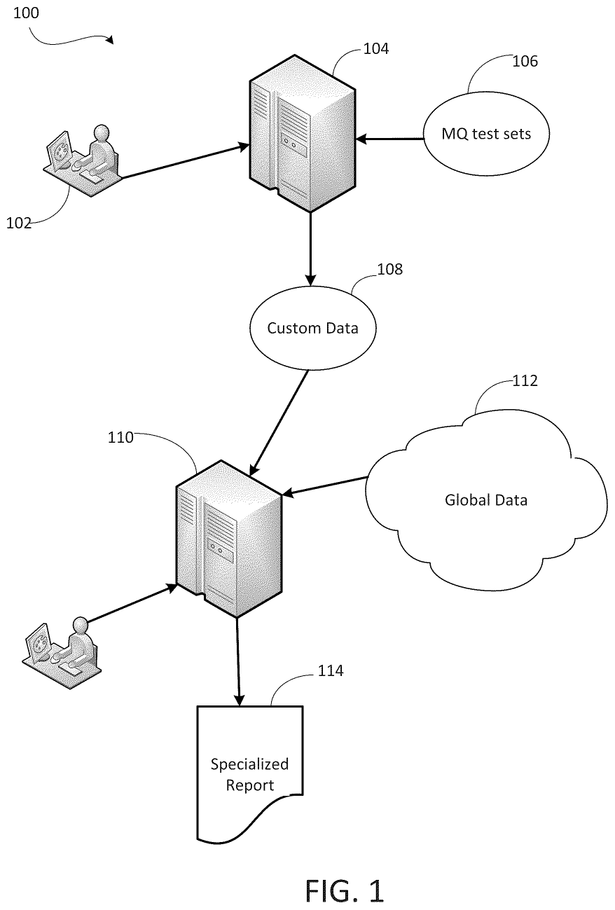

Therefore, an AI platform, combined with its unique MQ data, can help LLMs further analyze individual/group psychological conditions. FIG. 1 is an illustration of a LLMs process 100 according to the present invention. A user 102 interfaces with a MQ cloud platform 104. The MQ cloud platform 104 presents a collection of questions 106 to the user 102 and this collection of questions 106 is presented by a MQ test engine residing in the MQ cloud platform 104. The collection of questions 106 may be a collection of 100 plus questionnaires in different categories and the raw data from the user's response is processed by a MQ data analysis engine” Each of the categories may be related to a specific motivational factor. These factors are integral to understanding what drives an individual's behavior and preferences. The MQ data analysis engine will calculate a MQ value that represents the motivational value for the user. This MQ value is represented by a plurality of motivational factors, and through these factors, what motivates the user can be clearly identified. For example, a user may be more motivated when engaging in group activities and less motivated when assigned to a task that is performed by himself. Yet another user may be more motivated when giving a challenging problem and less motivated when assigned to a group activity. This MQ value is provided as part of a MQ report by a MQ report engine and presented to the user. This MQ value is subsequently fed to a MQ training LLMs engine.

The MQ training LLMs engine, which is also known as a web platform, takes user questions and uses these questions along with the information from the MQ report to “train” the MQ training LLMs engine. Training of this MQ training LLMs engine basically consists analyzing the MQ report and retrieving pertinent information related to the user questions. This training will result in a selection of a “user context template” and creation of a system command along with the MQ data to be sent to a specialized AI engine 110. This combination of the user context template, the system command, and the MQ data is also known as custom data 108. This specialized AI engine 110 interprets the system command and uses the user template to generate a user report 114 using the MQ data and the general global data 112 that the specialized AI engine has access.

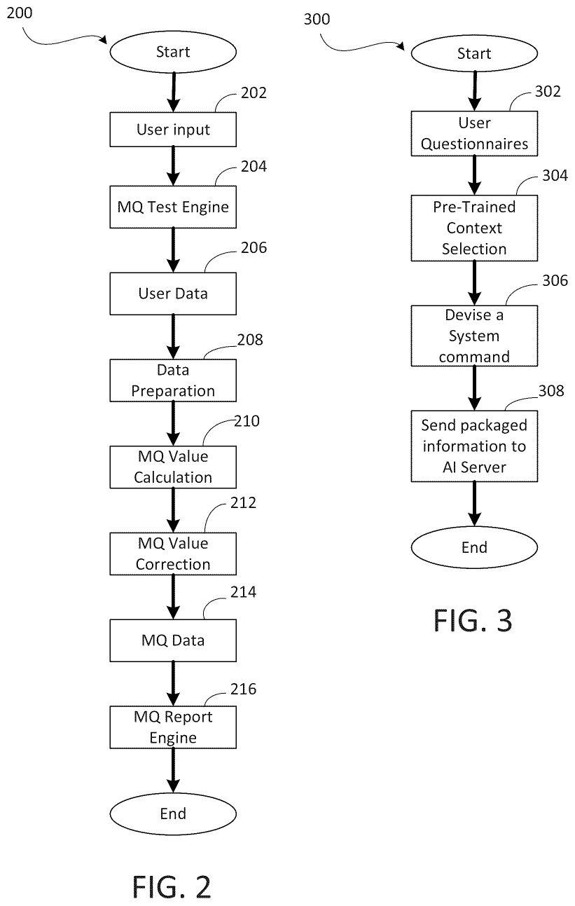

FIG. 2 is a MQ analysis process 200. User responds to a set of user questionnaires, step 202, and the user response is provided to a MQ test engine, step 204. The MQ test engine outputs data from the user response, step 206, and this user data is further analyzed and processed by the MQ data analysis engine. The user data comprises the user responses to a plurality set of questions and these questions may be classified into many categories. Some of the questions are repetitive in nature but asked in different ways. This user data will be further prepared, step 208. The data preparation includes grouping the user responses from similar categories and deriving an average value for each category, step 210. The result from the data preparation will be compared with a global data (data for general public) during a MQ value correction stage, step 212. Finally the MQ data is produced, step 214 and output by a MQ report engine, step 216. The MQ data is the context-data for the MQ training LLMs engine.

Once the MQ report (MQ data) is available to the user, the user may have question as how to interpret the information in the MQ report. The MQ training LLMs engine of the present invention will be able to assist the user to interpret the information in the MQ report. FIG. 3 illustrates a MQ training process 300. The MQ training process 300 starts with a web platform receiving a set of user questions, step 302, and from these questions interpreting the intent of the user and determining a context based on the user questions. Analyzing the user questions and the context data, the information related to user's motivation can be determined. From the context, a pre-trained context template is selected, step 304. For example, the user may want to know the information of his latest MQ report compared with his previous MQ report; his previous MQ report would be the context-data and the context would be comparison study. Alternatively, he may want to know his strength in the area of soft skills and for this situation his skills would be the context-data and strength analysis would be the context. The way the response to the user question is presented follows templates that have been pre “trained” by the MQ training LLMs engine. The MQ training LLMs engine pre train several templates based on the information from the MQ report and MQ training LLMs engine selects a template according to the user questions. Since the MQ report (MQ data) is related to user motivation, the selected template is also related the user motivation. A system command that instructs an AI engine how to respond to the user is devised, step 306. This system command and the selected template along with the motivation related data from the MQ report are sent to a LLM server, step 308. The steps 304, 306, and 308 are the essence of the “training” step. The LLM server now would have all the information regarding the user motivation and also the instruction how to interface with this user in addition to the all other data the AI engine has access to. This process according to the present invention enables the LLM server to interface more efficiently with the user.

When in use, the system of the present invention enables an AI engine to provide a tailored response to a user inquiry after the user has participated in a test to analyze his personal strength areas. The process starts with a user submitting his questions in the AI dialogue window, the system then activates the pre-trained context step in the training generated AI engine. For example, if the user wants to know the relationship analysis with another MQ test-taker, the system provides relevant training content to a LLM engine; if the user wants to know which professional career he should pursue, the system then provides a template that indicates what motivates the user along with the context data to the LLM engine. Choosing a career that matches one's motivation usually enables one to succeed in the chosen career.

The system selects the appropriate pre-trained context template related to the user's question. These contexts can include:
i. Historical Tracking Context: Tracking the user's multiple test results.
ii. Internal Analysis Context: Analyzing the user's intrinsic motivations and probable behaviors.
iii. Soft Skills Context: Analyzing the user's strengths in various soft skills.
iv. Team Comparison Context: Analyzing the motivational differences between the user and team members, potential conflicts, and suitability for team collaboration.
v. Comparison with Others Context: Analyzing the motivational differences between the user and group members, potential conflicts, and collaborative relationships.

Besides a pre-trained context, the system also organizes MQ data for LLMs training. The MQ data includes MQ values, which are calculated values and metrics from user's MQ assessment, and user data, which are relevant personal and contextual information from user's profile. The MQ data is an integration of the prepare data with pre-trained contexts, such that the LLMs engine has both the MQ data and the contextual information for analysis.

Finally, a system command is devised. In the final training step, LLMs is assigned a role and objective. For example, LLMs is instructed to act as an MQ coach whose goal is to help interpret MQ data. Language options are also added to ensure responses are in the preferred language (e.g., English).

The MQ data, the pre-trained context, and the system command are sent to the LLMs engine and the LLMs engine generates a response based on the information received. After the LLMs engine generates the response, the system receives it via API communication. The dialogue window then provides the organized response for the user to view.

In essence, the system of the present invention enables a user to submit answers to a set of question presented in an AI dialogue window of the system and the system will then activate the pre-trained context step. The system selects the relevant pre-trained context (e.g., historical tracking, internal analysis, soft skills, team comparison, and comparison with others). The user's MQ data is also reorganized and prepared for LLMs training; the MQ data includes MQ values and user data, which are integrated with the pre-trained contexts. Through the system command, LLMs is assigned a role (e.g., MQ coach) and given objectives, additionally language options are set to ensure responses are in the user's preferred language. After the LLMS engine generates the response, the system receives it via API communication and the dialogue window provides the organized response for the user to view.

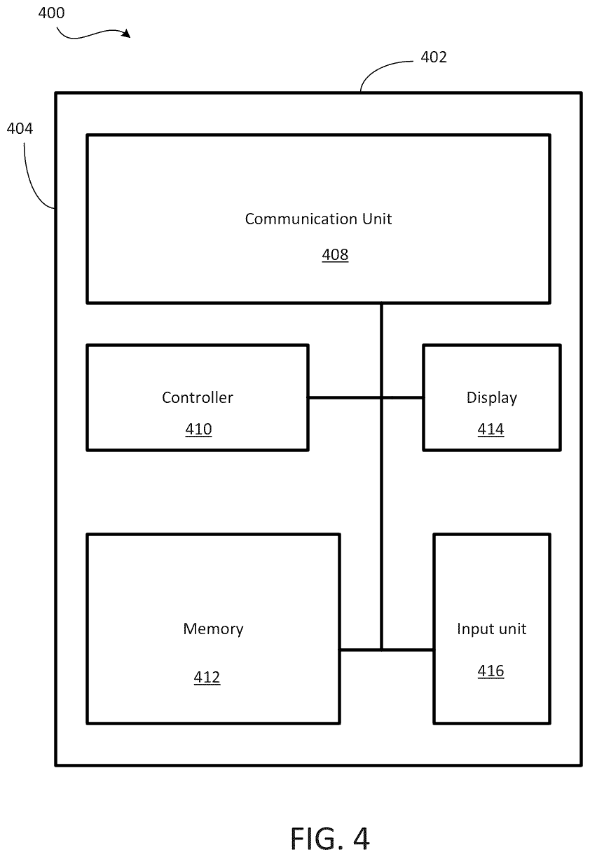

FIG. 4 illustrates an exemplary architecture 400 for a MQ training LLMs engine 402 of the present invention. The MQ training LLMs engine 402 has a communication unit 408 that enables the MQ training LLMs engine 402 to interface with an AI engine such as LLMs. The MQ training LLMs engine 402 further includes a controller 410, a display device 414, a memory 412, and a user interface unit 416. The display device 414 displays MQ data to the user. The user interface unit 416 enables a user to enter answers to the user questionnaires. The memory 412 is a non-transitory memory (a computer-readable medium) and capable of storing the MQ data and also the computer program instructions that support different features of the present invention. The controller 410 controls the operation of the MQ training LLMs engine. The processes described previously by FIGS. 1-3 are performed by the MQ training LLMs engine 402 executing the computer programs stored in the memory 412.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims. It is understood that features shown in different figures and described in different embodiments can be easily combined within the scope of the invention.

What is claimed is as follows:

1. A method for a web platform to train a large language model platform (LLM) to respond to user inquiries, the method comprising:
obtaining a context-data from a data analysis engine, the context-data being motivation factors for the user;
determining a context based on the context-data;
selecting a pre-trained context template for the context;
determining a system command for the LLMs platform; and
transmitting the context-data, the pre-trained context template, and the system command to the LLMs platform.

2. The method of claim 1 wherein the context-data further comprises personal psychological test data.

3. The method of claim 1 further comprising analyzing the context-data and retrieving pertinent information related to user motivation.

4. The method of claim 3 further comprising providing the user questions to the data analysis engine.

5. The method of claim 1 wherein determining a context based on the context-data further comprises comparing the context-data with a previous context-data for the user.

6. The method of claim 1 wherein the system command instructs the LLMs platform how to respond to the user.

7. A computer-readable medium on which is stored a computer program for an web platform to train a large language model platform to respond to an inquiry from a user, the computer program when executed by a computer, causes the web platform the steps for:
obtaining a context-data from a data analysis engine, the context-data being motivation factors for the user;
determining a context based on the context-data;
selecting a pre-trained context template for the context;
determining a system command for the LLMs platform;
transmitting the context-data, the pre-trained context template, and the system command to the LLMs platform.
